SEQUENCE LISTING (2 sequences)

SEQ ID NO: 1 - RNA, 23 nt, synthetic construct
  source 1..23: mol_type = other RNA
ctctcatcacggcacaacttagg

SEQ ID NO: 2 - RNA, 23 nt, synthetic construct
  source 1..23: mol_type = other RNA
taaagaggccactaaattgctgg

(19) United States
(12) Patent Application Publication
(10) Pub. No.: US 2024/0148831 A1
(43) Pub. Date: May 9, 2024

(54) COMBINATION TREATMENTS FOR CHOLESTASIS

(71) Applicant: THE BOARD OF REGENTS OF THE UNIVERSITY OF OKLAHOMA, Norman, OK (US)

(72) Inventor: Tiangang LI, Edmond, OK (US)

(73) Assignee: THE BOARD OF REGENTS OF THE UNIVERSITY OF OKLAHOMA, Norman, OK (US)

(21) Appl. No.: 18/501,014

(22) Filed: Nov. 2, 2023

Related U.S. Application Data
(60) Provisional application No. 63/382,161, filed on Nov. 3, 2022.

(51) Int. Cl. A61K 38/18; A61K 45/06; A61P 1/16

(52) U.S. Cl. CPC A61K 38/1825; A61K 45/06; A61P 1/16

(57) ABSTRACT

A method of treating a cholestasis liver condition in a subject by administering to the subject a first compound and a second compound, wherein the first compound is a fibroblast growth factor 19 (FGF19) analogue, and/or a bile acid-activated farnesoid x receptor (FXR) agonist, and the second compound is an apical sodium-dependent bile acid transporter (ASBT) inhibitor.

### CROSS-REFERENCE TO RELATED APPLICATIONS

The present application claims the benefit of U.S. Provisional Patent Application Ser. No. 63/382,161 filed Nov. 3, 2022, the disclosure of which is hereby expressly incorporated by reference in its entirety.

### STATEMENT REGARDING FEDERALLY SPONSORED RESEARCH AND DEVELOPMENT

This invention was made with government support under Grant No. DK117965 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Nov. 2, 2023, is named OKLAP0013US_ST26.xml and is 2,661 bytes in size.

### BACKGROUND

Bile acids are synthesized from cholesterol in hepatocytes and circulate between the liver and intestine in a process called the enterohepatic circulation. Bile acids act as physiological detergents to solubilize cholesterol in the bile and emulsify dietary lipids and fat-soluble vitamins in the small intestine. Bile acids also regulate a wide range of cellular pathways by activating nuclear receptors and cell surface receptors. In this regard, bile acid-activated farnesoid ×receptor (FXR) plays a key role in regulating bile acid homeostasis. Bile acids in hepatocytes activate FXR to feedback inhibit the transcription of the CYP7A1 gene, which encodes cholesterol 7α-hydroxylase (CYP7A1), the rate-limiting enzyme in de novo bile acid synthesis. Bile acids also activate intestinal FXR to induce mouse fibroblast growth factor 15 (FGF15) and human fibroblast growth factor 19 (FGF19), the human ortholog of FGF15, which inhibit the CYP7A1 gene and bile acid synthesis via a gut-liver endocrine axis. Mouse hepatocytes do not express FGF15, while human hepatocytes express FGF19, which is induced by hepatic FXR to inhibit CYP7A1 in an autocrine manner. These liver and intestine bile acid sensing mechanisms help maintain a relatively constant bile acid pool and prevent deleterious intracellular bile acid accumulation.

Both genetic and acquired impairment of bile flow out of the liver causes cholestasis, a pathological condition associated with intrahepatic bile acid accumulation leading to liver injury, inflammation, and fibrosis. The hydrophilic bile acid ursodeoxycholic acid (UDCA) and FXR agonist obeticholic acid (OCA) are approved therapies to treat a few forms of human cholestasis. However, therapeutic options are still limited for patients who do not adequately respond to these treatments. Because hydrophobic bile acid-induced damage to hepatocytes and bile duct epithelial cholangiocytes is a key pathogenic driver in cholestasis, an FGF19 analogue that inhibits bile acid synthesis and an inhibitor of apical sodium-dependent bile acid transporter (ASBT; also known as SLC10A2, and ileal bile acid transporter (IBAT)) that blocks intestine bile acid re-uptake have been tested separately as potential treatments for various forms of human cholestasis.

Due to the bile acid-mediated feedback regulation of bile acid synthesis and transport in the liver and the intestine, inhibition of de novo bile acid synthesis and a smaller bile acid pool decreases fecal bile acid loss, while blocking intestinal bile acid uptake causes compensatory induction of de novo bile acid synthesis. These compensatory mechanisms are expected to limit the magnitude of bile acid pool reduction that can be achieved by FXR agonists, FGF19 analogues, or ASBT inhibitors. Recently, it was reported that combining a gut restricted ASBT inhibitor GSK2330672 (GSK) with AAV-FGF15-mediated hepatocyte-specific FGF15 overexpression, which mimics FGF19 analogue therapy, effectively inhibited both hepatic bile acid synthesis and intestinal bile acid re-uptake, which achieved a significantly higher degree of bile acid pool reduction than either single treatment in WT mice. It was shown that in obese mice with non-alcoholic steatohepatitis, this combined treatment decreased gut cholesterol and fatty acid absorption, obesity, and hepatic steatosis and fibrosis. This co-treatment draws comparison to the FDA approved cholesterol lowering combination drug Vytorin (simvastatin/ezetimibe), in which simvastatin inhibits de novo cholesterol synthesis and ezetimibe inhibits intestinal cholesterol absorption. The recently completed clinical trial reports that by targeting both hepatic and intestinal sources of cholesterol Vytorin is significantly more effective in achieving the more stringent low-density lipoprotein-cholesterol lowering goal in type-2 diabetes patients and decreasing cardiovascular events and mortality in the high-risk population.

Many patients with various forms of genetic and acquired cholestasis could benefit from a higher degree of bile acid pool reduction than what is achievable by current therapies. It is to providing a therapy for cholestasis by lowering the bile acid pool in the liver that the present disclosure is directed.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B show that Cyp2c70 KO mice show enlarged hydrophobic bile acid pool than WT mice. WT and Cyp2c70 KO mice at 16 weeks of age were fasted for 6 h from 9 am to 3 pm and euthanized. Gallbladder bile acid pool composition is shown for males (A) and females (B). n=3-4. All results are expressed as mean±SEM. “*” indicates statistical significance (p<0.05, Student's t-test), vs. WT. ND: not detectable.

FIGS. 2A-2B show that Cyp2c70 KO mice show enlarged hydrophobic bile acid pool than WT mice. WT and Cyp2c70 KO mice at 16 weeks of age were fasted for 6 h from 9 am to 3 pm and euthanized. Tissue total bile acid content and total bile acid pool is shown for males (A) and females (B). n=5-8. All results are expressed as mean±SEM. “*” indicates statistical significance (p<0.05, Student's t-test), vs. WT. ND: not detectable.

FIGS. 3A-3B show that Cyp2c70 KO mice show enlarged hydrophobic bile acid pool than WT mice. WT and Cyp2c70 KO mice at 16 weeks of age were fasted for 6 h from 9 am to 3 pm and euthanized. Liver total bile acid concentration is shown for males (A) and females (B). n=5-8. G, H, I, J. Relative liver mRNA expression. n=8. All results are expressed as mean±SEM. “*” indicates statistical significance (p<0.05, Student's t-test), vs. WT. ND: not detectable.

FIGS. 4A-4B show that Cyp2c70 KO mice show enlarged hydrophobic bile acid pool than WT mice. WT and Cyp2c70 KO mice at 16 weeks of age were fasted for 6 h from 9 am to 3 pm and euthanized. Relative liver CYP7A1 mRNA expression is shown for males (A) and females (B). n=8. All results are expressed as mean±SEM. “*” indicates statistical significance (p<0.05, Student's t-test), vs. WT. ND: not detectable.

FIGS. 5A-5B show that Cyp2c70 KO mice show enlarged hydrophobic bile acid pool than WT mice. WT and Cyp2c70 KO mice at 16 weeks of age were fasted for 6 h from 9 am to 3 pm and euthanized. Relative liver CYP8B1 mRNA expression is shown for males (A) and females (B). n=8. All results are expressed as mean±SEM. “*” indicates statistical significance (p<0.05, Student's t-test), vs. WT. ND: not detectable.

FIG. 6A shows result for portal inflammation, ductular reaction and portal fibrosis in the male Cyp2c70 KO and WT mice. WT and Cyp2c70 KO mice at 16 weeks of age were fasted for 6 h from 9 am to 3 pm and euthanized. Representative images of immunohistochemistry of F4/80 stain and CK19 stain, and Sirius Red and H&E stain. p: portal vein; c: central vein. Scale bar=250 um. All results are expressed as mean±SEM. “*” indicates statistical significance (p<0.05, Student's t-test), vs. WT.

FIG. 6B shows result for portal inflammation, ductular reaction and portal fibrosis in the female Cyp2c70 KO and WT mice. WT and Cyp2c70 KO mice at 16 weeks of age were fasted for 6 h from 9 am to 3 pm and euthanized. Representative images of immunohistochemistry of F4/80 stain and CK19 stain, and Sirius Red and H&E stain. p: portal vein; c: central vein. Scale bar=250 um. All results are expressed as mean±SEM. “*” indicates statistical significance (p<0.05, Student's t-test), vs. WT.

FIGS. 7A-7B show results for portal inflammation, ductular reaction and portal fibrosis in the male and female Cyp2c70 KO and WT mice. WT and Cyp2c70 KO mice at 16 weeks of age were fasted for 6 h from 9 am to 3 pm and euthanized. Serum alanine aminotransferase (ALT) and aspartate aminotransferase (AST) is shown for males (A) and females (B). n=4-8. All results are expressed as mean±SEM. “*” indicates statistical significance (p<0.05, Student's t-test), vs. WT.

FIGS. 8A-8B show results for portal inflammation, ductular reaction and portal fibrosis in the male and female Cyp2c70 KO and WT mice. WT and Cyp2c70 KO mice at 16 weeks of age were fasted for 6 h from 9 am to 3 pm and euthanized. Relative liver COL1A1 and TIMP-1 mRNA expression for males (A) and females (B). n=6-13. All results are expressed as mean±SEM. indicates statistical significance (p<0.05, Student's t-test), vs. WT.

FIG. 9 shows therapeutic effects of GSK, AAV-FGF15 and the combined treatment in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. The Cyp2c70 KO mice in the “Control” group and the “GSK” group were injected with AAV-Null (1×10¹¹ GC/mouse). After 4 weeks, mice were fasted for 6 h from 9 am-3 pm and euthanized. Representative images of immunohistochemistry of F4/80 stain and CK19 stain, and Sirius Red and H&E stain are shown. Scale bar=250 um for F4/80, CK-19, and H&E; scale bar=600 um for Sirius Red. Age matched WT mice were included for comparison.

FIG. 10 shows therapeutic effects of GSK, AAV-FGF15 and the combined treatment in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. The Cyp2c70 KO mice in the “Control” group and the “GSK” group were injected with AAV-Null (1×10¹¹ GC/mouse). After 4 weeks, mice were fasted for 6 h from 9 am-3 pm and euthanized. Age matched WT mice were included for comparison. Results for serum alanine aminotransferase (ALT) and aspartate aminotransferase (AST) are shown. n=5-9. All results are expressed as mean±SEM. “*” indicates statistical significance (p<0.05, one way ANOVA and Dunnett's post hoc test), vs. Control.

FIGS. 11A-11B show therapeutic effects of GSK, AAV-FGF15 and the combined treatment in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. The Cyp2c70 KO mice in the “Control” group and the “GSK” group were injected with AAV-Null (1×10¹¹ GC/mouse). After 4 weeks, mice were fasted for 6 h from 9 am-3 pm and euthanized. Relative liver COL1A1 mRNA expression is shown in (A). Relative liver p21 mRNA expression is shown in (B). n=5-8. All results are expressed as mean±SEM. “*” indicates statistical significance (p<0.05, one way ANOVA and Dunnett's post hoc test), vs. Control.

FIG. 12 shows the effects of GSK, AAV-FGF15, and GSK+AAV-FGF15 treatment on bile acid metabolism in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are fecal total bile acids after 2 weeks of various treatments. n=3. All results are expressed as mean±SEM. “*” indicates statistical significance (p<0.05, one way ANOVA and Dunnett's post hoc test), comparison is either vs. “Control” or as indicated.

FIG. 13 shows the effects of GSK, AAV-FGF15, and GSK+AAV-FGF15 treatment on bile acid metabolism in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are tissue total bile acid content and total bile acid pool. n=5-8. All results are expressed as mean±SEM. “*” indicates statistical significance (p<0.05, one way ANOVA and Dunnett's post hoc test), comparison is either vs. “Control” or as indicated.

FIG. 14 shows the effects of GSK, AAV-FGF15, and GSK+AAV-FGF15 treatment on bile acid metabolism in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are relative liver CYP7A1 mRNA expression. n=5-8. All results are expressed as mean±SEM. “*” indicates statistical significance (p<0.05, one way ANOVA and Dunnett's post hoc test), comparison is either vs. “Control” or as indicated.

FIG. 15 shows the effects of GSK, AAV-FGF15, and GSK+AAV-FGF15 treatment on bile acid metabolism in the female Cyp2c70 KO mice. Female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results show percentages of various gallbladder bile acids n=3-5. All results are expressed as mean±SEM. “*” indicates statistical significance (p<0.05, one way ANOVA and Dunnett's post hoc test), comparison is either vs. “Control” or as indicated.

FIG. 16 shows the effects of GSK, AAV-FGF15, and the combined treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9am-3 pm and euthanized. Results are relative liver CYP8B1 mRNA expression. n=4-8. All results are expressed as mean±SEM. “*” indicates statistical significance (p<0.05, one way ANOVA and Dunnett's post hoc test), comparison is either vs. “Control” or as indicated otherwise.

FIG. 17 shows the effects of GSK, AAV-FGF15, and GSK+AAV-FGF15 treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are fecal total bile acids after 2 weeks of various treatments. n=3. All results are expressed as mean±SEM. “*” indicates statistical significance (p<0.05, one way ANOVA and Dunnett's post hoc test), comparison is either vs. “Control” or as indicated.

FIG. 18 shows the effects of GSK, AAV-FGF15, and GSK+AAV-FGF15 treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are tissue total bile acid content and total bile acid pool. n=5-8. All results are expressed as mean±SEM. “*” indicates statistical significance (p<0.05, one way ANOVA and Dunnett's post hoc test), comparison is either vs. “Control” or as indicated.

FIG. 19 shows the effects of GSK, AAV-FGF15, and GSK+AAV-FGF15 treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results are relative liver CYP7A1 mRNA expression. n=5-8. All results are expressed as mean±SEM. “*” indicates statistical significance (p<0.05, one way ANOVA and Dunnett's post hoc test), comparison is either vs. “Control” or as indicated.

FIG. 20 shows the effects of GSK, AAV-FGF15, and GSK+AAV-FGF15 treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9 am-3 pm and euthanized. Results show percentages of various gallbladder bile acids n=3-5. All results are expressed as mean±SEM. “*” indicates statistical significance (p<0.05, one way ANOVA and Dunnett's post hoc test), comparison is either vs. “Control” or as indicated.

FIG. 21 shows the effects of GSK, AAV-FGF15, and the combined treatment on bile acid metabolism in the male Cyp2c70 KO mice. Male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9am-3 pm and euthanized. Results are relative liver CYP8B1 mRNA expression. n=4-8. All results are expressed as mean±SEM. “*” indicates statistical significance (p<0.05, one way ANOVA and Dunnett's post hoc test), comparison is either vs. “Control” or as indicated otherwise.

FIGS. 22A-22B show that the combined treatment restored gut barrier integrity in the male and female Cyp2c70 KO mice. Male and female Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, mice were fasted for 6 h from 9am-3 pm and euthanized. A. Representative images of colon H&E stain. B. Representative images of colon immunohistochemistry stain of ZO-1. Scale bar=250 um.

FIGS. 23A-23B show that the combined treatment reduced fecal LCA abundance in the male and female Cyp2c70 KO mice. Female and male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, fresh feces were collected. Pooled fecal samples from 4 mice/group were used for all subsequent analyses. Results are fecal bile acid composition by percentage in females (A) and males (B). Results are expressed mean±SD of technical triplicates. “*” indicates statistical significance (p<0.05, one way ANOVA and Dunnett's post hoc test).

FIGS. 24A-24B show that the combined treatment reduced fecal LCA abundance in the male and female Cyp2c70 KO mice. Female and male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, fresh feces were collected. Pooled fecal samples from 4 mice/group were used for all subsequent analyses. Results are bile acid concentrations in fecal extracts in females (A) and males (B). “*” indicates statistical significance (p<0.05, one way ANOVA and Dunnett's post hoc test).

FIGS. 25A-25B show that the combined treatment reduced fecal LCA abundance in the male and female Cyp2c70 KO mice. Female and male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, fresh feces were collected. Pooled fecal samples from 4 mice/group were used for all subsequent analyses. Results show LC-MS measurement of T-CDCA-d4 transformation (ratio of CDCA-d4 to (T-CDCA-d4+CDCA-d4)) in fecal slurry as described in “Methods” for females (A) and males (B). Results are expressed mean±SD of technical triplicates. “*” indicates statistical significance (p<0.05, one way ANOVA and Dunnett's post hoc test).

FIGS. 26A-26B show that the combined treatment reduced fecal LCA abundance in the male and female Cyp2c70 KO mice. Female and male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, fresh feces were collected. Pooled fecal samples from 4 mice/group were used for all subsequent analyses. Results are LC-MS measurement of T-CDCA-d4 transformation (T-UDCA-d4 concentration) in fecal slurry as described in “Methods” for females (A) and males (B). Results are expressed mean±SD of technical triplicates. “*” indicates statistical significance (p<0.05, one way ANOVA and Dunnett's post hoc test).

FIGS. 27A-27B show that the combined treatment reduced fecal LCA abundance in the male and female Cyp2c70 KO mice. Female and male Cyp2c70 KO mice at 12 weeks of age were injected with AAV-FGF15 (1×10¹¹ GC/mouse) indicated as “FGF15”. Some mice were treated with GSK (5 mg/kg/day) indicated as “GSK”. Mice in the “Control” group and the “GSK” group were injected with AAV-Null (1×10¹¹ GC/mouse). After 4 weeks, fresh feces were collected. Pooled fecal samples from 4 mice/group were used for all subsequent analyses. Results are LC-MS measurement of T-CDCA-d4 transformation in fecal slurry as described in “Methods” for females (A) and males (B). The ratio of T-UDCA-d4 to the sum of T-CDCA-d4 and CDCA-d4 expressed as percentage. Results are expressed mean±SD of technical triplicates. “*” indicates statistical significance (p<0.05, one way ANOVA and Dunnett's post hoc test).

### DETAILED DESCRIPTION

Therapeutic reduction of hydrophobic bile acids is considered beneficial in treatments for cholestasis. The present disclosure describes a cholestasis combination therapy for lowering bile acid pool size and bile acid-associated hepatobiliary injury and fibrosis in all forms of genetic and acquired cholestasis condition by suppressing hepatic bile acid synthesis and intestinal bile acid uptake. The method of treating the cholestasis liver condition in a subject includes administering to the subject a first compound and a second compound, wherein the first compound may be a fibroblast growth factor 15 (FGF15) analogue, a fibroblast growth factor 19 (FGF19) analogue, and/or a bile acid-activated farnesoid ×receptor (FXR) agonist, and the second compound is an apical sodium-dependent bile acid transporter (ASBT) inhibitor. In certain embodiments the FGF19 analogue is Aldafermin (NGM282). In certain embodiments, the FXR agonist is selected from Obeticholic acid (Ocaliva), Tropifexor, Cilofexor, EDP-305, EDP-297, and MET409. In certain embodiments, the ASBT inhibitor is selected from Linerixibat (GSK2330672), Lopixibat, Volixibat (SHP626), Odevixibat (A4250), Barixibat, Elobixibat (A3309), SC-435, S-1647, IMB17-15, 264W94, S-8921, S-8921G, R-146224, BRL-39924A, and S 0960. The first compound and the second compound may interact synergistically. The first compound may be administered before, concurrently with, or after the second compound is administered.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art.

The following abbreviations may be used herein:
AAV: Adeno-associated virus;
ALT: alanine aminotransferase;
ANOVA: Analysis of variance;
ASBT: apical sodium-bile acid transporter;
AST: aspartate aminotransferase;
CA: cholic acid;
CDCA: chenodeoxycholic acid;
CK-19: cytokeratin-19:
CYP7A1: Cholesterol 7a-hydroxylase;
CYP8B1: sterol 12a-hydroxylase;
DCA: deoxycholic acid;
FGF-15: fibroblast growth factor-15;
FXR: farnesoid ×receptor;
GSK: GSK2330672;
H&E: hematoxylin and eosin;
IBAT: ileal bile acid transporter;
KO: knockout;
LC-MS: Liquid chromatography-mass spectrometry;
LCA: lithocholic acid;
MCAs: muricholic acids;
mRNA: messenger ribonucleic acid;
ND: not detectable;
OCA: obeticholic acid;
SEM: Standard error of the mean;
T-CDCA-d4: tauro-CDCA-d4;
T-LCA tauro-lithocholic acid;
UDCA: ursodeoxycholic acid;
WT: wild type;
ZO-1: Zonula occludens-1;
7α-HSDH: 7α-hydroxysteroid dehydrogenase; and
7β-HSDH: 7 β-hydroxysteroid dehydrogenase.

As utilized in accordance with the methods, compounds, and compositions of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. Further, unless otherwise required by context, singular terms shall include pluralities and plural terms shall include the singular. Where used herein, the specific term “single” is limited to only “one”.

Where used herein, the pronoun “we” is intended to refer to all persons involved in a particular aspect of the investigation disclosed herein and as such may include non-inventor laboratory assistants and collaborators working under the supervision of the inventor.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc., all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the active agent or composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

By “biologically active” is meant the ability of the active agent to modify the physiological system of an organism without reference to how the active agent has its physiological effects. The effectiveness of a method or use, such as a treatment that provides a potential therapeutic benefit or improvement of a condition or disease, can be ascertained by various methods and testing assays.

The active agents which comprise the combination treatments disclosed herein can be used in treatments of various cholestasis liver conditions, including but not limited to Primary biliary cholangitis, Primary sclerosing cholangitis, Biliary atresia, Progressive familial intrahepatic cholestasis 1 (PFIC 1), Progressive familial intrahepatic cholestasis 2 (PFIC 2), Progressive familial intrahepatic cholestasis 3 (PFIC 3), and Progressive familial intrahepatic cholestasis 4 (PFIC 4).

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically acceptable excipients, including carriers, vehicles, diluents, and adjuvants which may improve solubility, deliverability, dispersion, stability, and/or conformational integrity of the compounds or conjugates thereof.

The term “active agent” as used herein refers to analogues of FGF15 and FGF19, agonists of FXR, and inhibitors of ASBT. In some embodiments the active agents are conjugated to a targeting moiety to form a conjugate. A conjugate is a compound comprising an active agent covalently linked, directly or indirectly via a linker molecule, to a secondary compound, such as an antibody or fragment thereof. The active agent may be associated with a targeting moiety or molecule which is able to bind to a target cell such as a hepatocyte or a portion of a target cell. The targeting moiety may be linked directly or indirectly to the active agent, or to the pharmaceutically acceptable carrier, vehicle, or diluent which contains or is associated with the active agent. The targeting moiety may be any molecule that can bind to another molecule. For example, a targeting moiety may include an antibody or its antigen-binding fragments, a receptor molecule, a chimeric antibody molecule, or an affinity reagent. As used herein, the term “targeting moiety” refers to a structure that binds or associates with a biological moiety or fragment thereof. As noted, in some embodiments, the targeting moiety may be an antibody. In some embodiments, the targeting moiety may be a monoclonal antibody (mAb). In some embodiments, the targeting moiety may be an antibody fragment, surrogate, or variant. In some embodiments, the targeting moiety may be a protein ligand. In some embodiments, the targeting moiety may be a protein scaffold. In some embodiments, the targeting moiety may be a peptide. In some embodiments, the targeting moiety may be RNA or DNA. In some embodiments, the targeting moiety may be a RNA or DNA fragment. In some embodiments, the targeting moiety may be a small molecule ligand.

In at least certain compounds of the present disclosure, polyethylene glycol (PEG) molecules (also known as poly(ethylene oxide) and poly(oxyethylene)) are used, for example as linkers to link other compounds together to for drug conjugates. PEG comprises repeating units of ethylene glycol and is available in different average molecular weights (MW) based on the average number of ethylene glycol units in the PEG molecules of the particular PEG composition. For example, PEG₈₈, a PEG molecule with 2 ethylene glycol units, has a MW of 88 Daltons (Da). PEG₄₀₀, a PEG molecule with about 8 ethylene glycol units, has a MW of 400 Daltons (Da). PEG_{60,000}, a PEG molecule with about 1364 ethylene glycol units, has a MW of about 60,000. The PEG molecule may comprise up to 30,000 ethylene glycol units, Other examples include, but are not limited to, PEG₂₀₀ having an average MW of about 200 Daltons (Da), PEG₃₀₀ having an average MW of about 300 Da, PEG₄₀₀ having an average MW of about 400 Da, PEG₅₀₀ having an average MW of about 500 Da, PEG₇₅₀ having an average MW of about 750 Da, PEG₁₀₀₀ having an average MW of about 1000 Da, PEG₁₅₀₀ having an average MW of about 1500 Da, PEG₂₀₀₀ having an average MW of about 2000 Da, PEG₃₀₀₀ having an average MW of about 3000 Da, PEG₃₃₅₀ having an average MW of about 3350 Da, PEG₃₅₀₀ having an average MW of about 3500 Da,PEG₄₀₀₀ having an average MW of about 4000 Da, PEG₅₀₀₀ having an average MW of about 5000 Da, PEG₆₀₀₀ having an average MW of about 6000 Da, PEG₇₅₀₀ having an average MW of about 7500 Da, PEG_{10,000} having an average MW of about 10,000 Da, PEG_{15,000} having an average MW of about 15,000 Da, PEG_{20,000} having an average MW of about 20,000 Da, PEG_{25,000} having an average MW of about 25,000 Da, PEG_{30,000} having an average MW of about 30,000 Da, PEG_{40,000} having an average MW of about 40,000 Da, PEG_{50,000} having an average MW of about 50,000 Da, and PEG_{60,000} having an average MW of about 60,000 Da. Where used herein the term PEG is intended to refer to any of the examples of PEG listed above, and to PEGs having MWs in the range of 88 and 60,000, unless a particular MW is specified. In other embodiments, the linker molecule may be an amino acid, a peptide, or a polypeptide,

In various non-limiting embodiments, the drug conjugates of the present disclosure include active agents which are linked via a linker (e.g., a PEG, amino acid, peptide or polypeptide) to an anchor-solubilizing moiety such as a phosphatidylethanolamine (PE). Examples of such PEs include but are not limited to distearoylphosphatidylethanolamine (DSPE), dipalmitoylphosphatidylethanolamine, dimyristoylphosphatidylethanolamine, diarachidylphosphatidylethanolamine, dilaurylphosphatidylethanolamine, dioleylphosphatidylethanolamine, palmitoylstearoylphosphatidylethanolamine, myristoylstearoylphosphatidylethanolamine, arachidylstearoylphosphatidylethanolamine, laurylstearoylphosphatidylethanolamine, oleylstearoylphosphatidylethanolamine, myristoylpalmitoylphosphatidylethanolamine, arachidylpalmitoylphosphatidylethanolamine, laurylpalmitoylphosphatidylethanolamine, arachidylmyristoylphosphatidylethanolamine, laurylmyristoylphosphatidylethanolamine, laurylarachidylphosphatidylethanolamine, oleylpalmitoylphosphatidylethanolamine, oleylmyristoylphosphatidylethanolamine, oleylarachidylphosphatidylethanolamine, and lauryloleylphosphatidylethanolamine,. In other embodiments, anchoring/solubilizing moiety may comprise any one of the above moieties wherein the ethanolamine is substituted with serine (forming a phosphatidylserine (PS)) or choline (forming a phosphatidylcholine (PC)), such as distearoylphosphatidylserine or distearoylphosphatidylcholine. In other embodiments, the anchoring/solubilizing moiety may comprise a combination of two or more of the above moieties. In other embodiments, anchoring/solubilizing moiety may comprise a single saturated, unsaturated, or polyunsaturated lipid molecule comprising 2-28 carbon atoms, particularly 10-18 carbon atoms, such as a saturated, unsaturated, or polyunsaturated fatty acid. The anchor-solubilizing moiety may comprise a PE, PS or PC with a single fatty acid or two fatty acids, which may be selected from the group of saturated, unsaturated, and polyunsaturated fatty acids.

In particular, non-limiting examples, the targeting vector of the present disclosure is combined with liposomes in which a cargo molecule is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the active agent to be delivered. Liposomes can be made from phospholipids other than naturally derived phosphatidylcholine. Neutral liposome compositions, for example, can be formed from dimyristoyl phosphatidylcholine (DMPC) or dipalmitoyl phosphatidylcholine (DPPC) or other similar lipids. Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

The term “antibody” as used herein can refer to both intact “full length” antibodies as well as to antigen-binding fragments thereof (unless otherwise explicitly noted). The afore-mentioned antigen-binding fragments may also be referred to herein as antigen binding fragments, antigen binding compounds, antigen binding portions, binding fragments, binding portions, or antibody fragments. Also, as used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)2 fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}—C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, monoclonal antibodies can be made by the hybridoma method first described by Kohler et al. (Nature, 256:495 (1975)), or may be made by recombinant DNA methods (see, for example, U.S. Pat. No. 4,816,567).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies and antigen binding compounds described herein can be provided in isolated and/or purified form.

In at least certain embodiments of the present disclosure, the term “therapeutic agent” refers to an active agent comprising an antibody and/or antibody-derived compound or other compound as described herein.

A “diagnostic agent,” which may also be referred to herein as an imaging agent, is a substance that is useful in diagnosing a disease or imaging a cell or tissue. Useful diagnostic agents of the present disclosure may include antibodies and antibody-derived compounds described herein, and may further comprise by linkage or other association radioisotopes, dyes, contrast agents, fluorescent compounds or molecules, and enhancing agents (e.g., paramagnetic ions).

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the active agent to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the active agent of the present disclosure may be designed to provide delayed, controlled, extended, and/or sustained release using formulation techniques which are well known in the art.

The term “topical” is used herein to define a mode of administration through an internal or external epithelial surface, such as but not limited to, a material that is administered by being applied externally to the eye or a nasal mucosa. A non-limiting example of topical administration is through the use of eyedrops or through the use of a nasally administered aerosol.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of the active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As noted above, the combinations of the active agents of the present disclosure, such as an FGF19 analogue and an ASBT inhibitor, or an FXR agonist and an ASBT inhibitor, may act synergistically. As used herein the terms “synergism,” “synergistic,” or “synergistic effect” refers to a therapeutic effect or result that is greater than the additive effects of each active agent used individually. Presence or absence of a synergistic effect for a particular combination of treatment substances can be quantified, for example, by using the Combination Index (CI) (e.g., Chou, Pharmacol Rev, 2006. 58(3): 621-81), wherein CI values lower than 1 indicate synergy and values greater than 1 imply antagonism. Combinations of the inhibitors and antagonists of the present disclosure can be tested in vitro for synergistic cell growth inhibition using standard cell lines for particular cancers, or in vivo using standard animal cancer models. A synergistic effect of a combination described herein can permit, in some embodiments, the use of lower dosages of one or more of the components of the combination. A synergistic effect can also permit, in some embodiments, less frequent administration of at least one of the administered active agents. Such lower dosages and reduced frequency of administration can reduce the toxicity associated with the administration of at least one of the therapies to a subject without reducing the efficacy of the treatment.

The term “coadministration” refers to administration of two or more active agents, e.g., an FGF19 analogue and an ASBT inhibitor, or an FXR agonist and an ASBT inhibitor. The timing of coadministration depends in part of the combination and compositions administered and can include administration at the same time (i.e., concurrently), before, just prior to, or after, or just after the administration of one or more additional therapies Coadministration is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating bile acid conditions co-occurring conditions thereof.

The active agents of the present disclosure may be present in the pharmaceutical compositions at any concentration that allows the pharmaceutical composition to function in accordance with the present disclosure; for example, but not by way of limitation, the active agents may be present in the composition in a range having a lower level selected from 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure.

Suitable carriers, vehicles, and other components that may be included in the formulation are described, for example, in Remington: The Science and Practice of Pharmacy, 21^{st}Ed. and 22^{nd} Ed. The term “pharmaceutically acceptable” means that the carrier is a non-toxic material that does not interfere with the effectiveness of the biological activity of the active agent. The characteristics of the carrier will depend on various factors, including but not limited to, the route of administration.

For example, but not by way of limitation, the active agent may be dissolved in a physiologically acceptable pharmaceutical carrier or diluent and administered as either a solution or a suspension. Non-limiting examples of suitable pharmaceutically acceptable carriers include water, saline, dextrose solutions, fructose solutions, ethanol, or oils of animal, vegetative, or synthetic origin, or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the active agent and pharmaceutically acceptable carrier(s) (and other additional therapeutically active agent(s), if present). Examples of additional components that may be present include, but are not limited to, diluents, fillers, salts, buffers, preservatives, stabilizers, solubilizers, and other materials well known in the art. Another particular non-limiting example of an additional component that may be present in the pharmaceutical composition is a delivery agent, as discussed in further detail herein below.

Other embodiments of the pharmaceutical compositions of the present disclosure may include the incorporation or entrapment of the active agent in various types of drug delivery systems that function to provide targeted delivery, controlled release, and/or increased half-life to the active agent. For example, but not by way of limitation, it is possible to entrap the active agent in microcapsules prepared by coacervation techniques or by interfacial polymerization (for example, hydroxymethylcellulose or gelatin-microcapsules and poly-(methylmethacylate) microcapsules, respectively). It is also possible to entrap the active agent in macroemulsions or colloidal drug delivery systems (such as but not limited to, liposomes, albumin microspheres, microemulsions, nanoparticles, nanocapsules, and the like). Such techniques are well known to persons having ordinary skill in the art, and thus no further description thereof is deemed necessary.

In one particular, non-limiting example, the pharmaceutical composition may include a liposome in which the active agent is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference.

In other non-limiting examples, the active agent of the present disclosure may be incorporated into particles of one or more polymeric materials, as this type of incorporation can be useful in controlling the duration of action of the active agent by allowing for controlled release from the preparations, thus increasing the half-life thereof. Non-limiting examples of polymeric materials that may be utilized in this manner include polyesters, polyamides, polyamino acids, hydrogels, poly(lactic acid), ethylene vinylacetate copolymers, copolymer micelles of, for example, PEG and poly(1-aspartamide), and combinations thereof.

The pharmaceutical compositions described or otherwise contemplated herein may further comprise at least one delivery agent, such as a targeting moiety, that assists in delivery of the active agent to a desired site of delivery, such as a liver hepatocyte.

The compositions of the present disclosure may be formulated for administration by any other method known or otherwise contemplated in the art, as long as the route of administration allows for delivery of the active agent so that the compounds can function in accordance with the present disclosure, e.g., to reduce cholestasis. Examples of other routes of administration include, but are not limited to, oral, topical, retrobulbar, subconjunctival, transdermal, parenteral, subcutaneous, intranasal, intramuscular, intraperitoneal, intravitreal, and intravenous routes, including both local and systemic application routes.

Another non-limiting embodiment of the present disclosure is directed to a kit that contain one or more of any of the pharmaceutical compositions described or otherwise contemplated herein. The kit may further contain a second agent as described herein above for use concurrently with the pharmaceutical composition(s). If the composition present in the kit is not provided in the form in which it is to be delivered, the kit may further contain a pharmaceutically acceptable carrier, vehicle, diluent, or other agent for mixing with the active agent for preparation of the pharmaceutical composition. The kit including the composition and/or other reagents may also be packaged with instructions packaged for administration and/or dosing of the compositions contained in the kit. The instructions may be fixed in any tangible medium, such as printed paper, or a computer-readable magnetic or optical medium, or instructions to reference a remote computer data source such as a worldwide web page accessible via the internet.

The kit may contain single or multiple doses of the pharmaceutical composition which contains the active agent. When multiple doses are present, the doses may be disposed in bulk within a single container, or the multiple doses may be disposed individually within the kit; that is, the pharmaceutical compositions may be present in the kit in unit dosage forms to facilitate accurate dosing. The term “unit dosage forms” as used herein refers to physically discrete units suitable as unitary dosages for human subjects and other mammals; each unit contains a predetermined quantity of the active agent calculated to produce the desired therapeutic effect, in association with a suitable pharmaceutical excipient. Typical unit dosage forms of liquid compositions include prefilled, premeasured ampules or syringes; for solid compositions, typical unit dosage forms include pills, tablets, capsules, or the like. In such compositions, the active agent may sometimes be a minor component (from about 0.1 to about 50% by weight, such as but not limited to, from about 1 to about 40% by weight) with the remainder being various vehicles or carriers and processing aids helpful for forming the desired dosing form.

The active agent may be provided as a “pharmaceutically acceptable salt,” which refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297 (incorporated by reference herein in its entirety).

The amount of the active agent that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases or other conditions involved; the degree, involvement, and/or severity of the diseases or conditions; the response of the individual subject; the particular active agent administered; the mode of administration; the bioavailability characteristics of the preparation administered; the dose regimen selected; the use of concomitant medication; and other relevant circumstances. A therapeutically effective amount of an active agent of the present disclosure also refers to an amount of the active agent which is effective in controlling, reducing, or ameliorating the condition to be treated.

Practice of the method of the present disclosure may include administering to a subject a therapeutically effective amount of the pharmaceutical composition (containing the active agent in any suitable systemic and/or local formulation, in an amount effective to deliver the dosages listed above. The dosage can be administered, for example, but not by way of limitation, on a one-time basis, or administered at multiple times (for example, but not by way of limitation, from one to five times per day, or once or twice per week). The pharmaceutical composition may be administered either alone or in combination with other therapies, in accordance with the inventive concepts disclosed herein.

Compositions of the active agent can be administered in a single dose treatment or in multiple dose treatments on a schedule and over a time period appropriate to the age, weight and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, or whether the composition is used for prophylactic or curative purposes. For example, in certain embodiments, the composition is administered once per month, twice per month, three times per month, every other week, once per week, twice per week, three times per week, four times per week, five times per week, six times per week, every other day, daily, twice a day, or three times a day. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) or vehicle to form a pharmacological composition. Pharmaceutically acceptable carriers can contain a physiologically acceptable compound that acts to, e.g., stabilize, or increase or decrease the absorption or clearance rates of the pharmaceutical compositions. Physiologically acceptable carriers and vehicles can include, for example, carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds, carriers, and vehicles include wetting agents, emulsifying agents, dispersing agents or preservatives.

When administered orally, the present compositions may be protected from digestion. This can be accomplished either by complexing the active agent with a composition to render it resistant to acidic and enzymatic hydrolysis or by packaging active agent in an appropriately resistant carrier such as a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical, transdermal administration, the agents are formulated into ointments, creams, salves, powders and gels. Transdermal delivery systems can also include, e.g., patches. The present compositions can also be administered in sustained delivery or sustained release mechanisms. For example, biodegradeable microspheres or capsules or other biodegradeable polymer configurations capable of sustained delivery of the active agent can be included herein.

For inhalation, the active agent can be delivered using any system known in the art, including dry powder aerosols, liquids delivery systems, air jet nebulizers, propellant systems, and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

The active agent can be delivered alone or as pharmaceutical compositions by any means known in the art, e.g., systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the pharmaceutical formulations comprising the active agent are incorporated in lipid monolayers or bilayers, e.g., liposomes, such as shown in U.S. Pat. Nos. 6,110,490; 6,096,716; 5,283,185; and 5,279,833. Liposomes and liposomal formulations can be prepared according to standard methods and are also well known in the art, such as U.S. Pat. Nos. 4,235,871; 4,501,728 and 4,837,028.

In one aspect, the active agent is prepared with one or more carriers that will protect the active agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The active agent in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one example of a pharmaceutically suitable excipient.

Examples of routes of administration of the active agents described herein include parenteral injection, e.g., by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Formulated compositions comprising the active agent can be used for subcutaneous, intramuscular or transdermal administration. Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The active agents may be administered in solution. The formulation thereof may be in a solution having a suitable pharmaceutically acceptable buffer such as phosphate, Tris (hydroxymethyl) aminomethane-HCl or citrate, and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

For example, but not by way of limitation, the therapeutically effective amount of an active agent used in the present disclosure will generally contain sufficient active agent to deliver in a range of from about 0.01 μg/kg to about 10 mg/kg (weight of active agent/body weight of patient). For example, but not by way of limitation, the composition will deliver about 0.1 μg/kg to about 5 mg/kg, and more particularly about 1 μg/kg to about 1 mg/kg.

Exemplary, non-limiting ranges for a therapeutically or prophylactically effective amount of the active agent include but are not limited to 0.001 mg/kg of the subject's body weight to 100 mg/kg of the subject's body weight, more typically 0.01 mg/kg to 100 mg/kg, 0.1 mg/kg to 50 mg/kg, 0.1 mg/kg to 40 mg/kg, 1 mg/kg to 30 mg/kg, or 1 mg/kg to 20 mg/kg, or 2 mg/kg to 30 mg/kg, 2 mg/kg to 20 mg/kg, 2 mg/kg to 15 mg/kg, 2 mg/kg to 12 mg/kg, or 2 mg/kg to 10 mg/kg, or 3 mg/kg to 30 mg/kg, 3 mg/kg to 20 mg/kg, 3 mg/kg to 15 mg/kg, 3 mg/kg to 12 mg/kg, or 3 mg/kg to 10 mg/kg, or 5 mg to 1500 mg, as a fixed dosage.

The composition is formulated to contain an effective amount of the active agent, wherein the amount depends on the animal to be treated and the condition to be treated. In certain embodiments, the active agent is administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 pg to about 300 pg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

The dosage of an administered active agent for humans will vary depending upon such factors as the patient's age, weight, height, sex, general medical condition and previous medical history. In certain non-limiting embodiments, the recipient is provided with a dosage of the active agent that is in the range of from about 1 mg to 1000 mg as a single infusion or single or multiple injections, although a lower or higher dosage also may be administered. The dosage may be in the range of from about 25 mg to 100 mg of the active agent per square meter (m²) of body surface area for a typical adult, although a lower or higher dosage also may be administered. Examples of dosages that may be administered to a human subject further include, for example, 1 to 500 mg, 1 to 70 mg, or 1 to 20 mg, although higher or lower doses may be used. Dosages may be repeated as needed, for example, once per week for 4-10 weeks, or once per week for 8 weeks, or once per week for 4 weeks. It may also be given less frequently, such as every other week for several months, or more frequently, such as twice weekly or by continuous infusion.

CYP2C70, which is encoded by the Cyp2c70 gene, mediates the synthesis of muricholic acids (MCAs) primarily from chenodeoxycholic acid (CDCA) in mice and is responsible for the species difference of primary bile acids in humans and mice. In comparison to human bile acid pool, the significantly less hydrophobic bile acid pool due to the presence of MCAs in mice has been a limitation of investigating bile acid-induced hepatobiliary toxicity and the pathological impact of altered bile acid composition observed in WT mice cannot be extrapolated to humans. However, the bile of Cyp2c70 knockout (KO) mice contains primarily hydrophobic CDCA and cholic acid (CA), thus these mice exhibit a more human-like hydrophobic bile acid pool-induced hepatobiliary injury phenotype. The therapeutic benefits of combining an apical sodium-dependent bile acid transporter (ASBT) inhibitor GSK2330672 (GSK) and AAV-FGF15 in alleviating hepatobiliary bile acid toxicity in the Cyp2c70 knockout (KO) mice were investigated. The work revealed differential impacts of GSK, AAV-FGF15, and the combined GSK+AAV-FGF15 treatment on bile acid pool size and composition and the therapeutic efficacy toward cholangiopathy, portal fibrosis, and gut barrier integrity in male and female Cyp2c70 KO mice.

In certain embodiments of the present disclosure, the combination treatment comprises administration of an FGF15 or FGF19 analogue (i.e. NGM282) with an ASBT inhibitor. Other FGF19 analogues are disclosed in US. Patent Application Publication 2019/0175693, the entirety of which is hereby expressly incorporated herein by reference. In other embodiments of the present disclosure, the combination treatment comprises administration of an FXR agonist with an ASBT inhibitor. The combination treatment causes simultaneous inhibition of hepatic bile acid synthesis and intestinal bile acid absorption. By preventing the compensatory induction of hepatic bile acid synthesis associated with ASBT inhibitor monotherapy while simultaneously promoting fecal bile excretion, combining the two drugs can decrease total bile acid pool and toxicity to a higher degree than either single treatment, therefore achieving enhanced therapeutic efficacy in cholestasis.

Examples of types of acquired or genetic cholestasis liver diseases or cholestasis-related liver diseases that can be treated using the combination treatments disclosed herein include, but are not limited to, (1) Primary biliary cholangitis, (2) primary sclerosing cholangitis, (3) biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

The following are non-limiting examples of dosages of FGF15 or FGF19 analogues and ASBT compounds that can be used in these treatments:
FGF19 analogue (Aldafermin):
1 mg, 3 mg or 6 mg once daily injection.

ASBT inhibitors:
(a) A4250: 0.75, 1.5 or 3 mg/day.
(b) Volixibat (SHP626): 0.5, 1, 5, 10, or 20 mg/day.
(c) GSK2330672: 45 mg or 90 mg/day.
(d) 264W94: 45 mg or 90 mg/day.
(e)SC-435: 50-100 mg/day.
(f) A3309 (elobixibat): at 10 mg/day.

The following are non-limiting examples of dosages of FXR agonists and ASBT compounds that can be used in these treatments:

1. FXR agonists:
(a) Obeticholic acid (Ocaliva): 5 mg or 10 mg/day dose.
(b) Tropifexor: 60-200 ug/day.
(c) Cilofexor: 30 or 100 mg/day.
(d) EDP-305: 1 or 2.5 mg/day.
(e) MET-409: 50 or 80 mg/day.
   2. ASBT inhibitors:
(a) A4250: 0.75, 1.5 or 3 mg/day.
(b) Volixibat (SHP626): 0.5, 1, 5, 10, or 20 mg/day.
(c)GSK2330672: 45 mg or 90 mg/day.
(d) 264W94: 45 mg or 90 mg/day.
(e)SC-435: 50-100 mg/day.
(f) A3309 (elobixibat): at 10 mg/day.

### EXAMPLES

Examples are provided hereinbelow. However, the present disclosure is to be understood as not limited in its application to the specific experimentation, results, and laboratory procedures disclosed below or elsewhere herein. Rather, each example is provided as one of various embodiments and are meant to be exemplary, not exhaustive.

EXPERIMENTAL

Methods

Reagents

AST and ALT assay kits were purchased from Pointe Scientific (Canton. MI).

GSK2330672 was purchased from MedChemExpress LLC (Monmouth Junction, NJ). Bile acid assay kit was purchased from Diazyme Laboratories (Poway, CA). AAV8-ALB-FGF15 (under the control of an albumin promoter) was purchased from GeneCopoeia, Inc. (Rockville, MD). AAV-Null was purchased from Vector Biolabs Inc. (Malvern, PA). F4/80 antibody (Cat #. 70076) was purchased from Cell Signaling Technology (Danvers, MA). CK19 antibody (ab52625) was purchased from Abcam (Waltham, MA). ZO-1 antibody (PA5-28858) was purchased from ThermoFisher Scientific (Grand Island, NY).

Mice

The Cyp2c70 KO mice with Exon 4 deletion were generated by CRISPR/Cas-mediated genome engineering. Cas9 and gRNA (gRNA1 matching reverse strand of the Cyp2c70 gene: CTCTCATCACGGCACAACTTAGG (SEQ ID NO:1); gRNA2 matching forward strand of the Cyp2c70 gene: TAAAGAGGCCACTAAATTGCTGG (SEQ ID NO:2)) were co-injected into fertilized eggs of C57BL/6J mice. One F0 male founder mouse was identified by PCR followed by sequencing analysis to confirm deletion of an 888 bp genome region containing the entire Exon 4. This F0 founder was bred to a WT C57BL/6J mouse to obtain 3 F1 Cyp2c70+/−females confirming germline transmission. Further breeding of the F1 females with WT C57BL/6J mice (the Jackson Lab, Bar Harbor, ME) yielded WT mice and Cyp2c70+/−mice. Subsequent breeding of the Cyp2c70+/−offspring yielded WT mice and Cyp2c70−/− mice (Cyp2c70 KO). The Cyp2c70 KO mice were then bred via KO×KO breeding scheme. The WT littermates were used to generate WT mice via WT×WT breeding scheme. The Cyp2c70 KO pups weaned at 3 weeks of age showed significantly increased mortality, which was largely prevented when they were weaned at 4 weeks of age. Mice were housed in micro-isolator cages with Biofresh performance bedding (Pelleted cellulose) under 7 AM-7 PM light cycle and 7 PM-7 AM dark cycle. GSK was mixed with chow diet to achieve an estimated 5 mg/kg/day intake based on 5 g/day food intake by a 25 g mouse. AAV-Null or AAV-FGF15 was injected via tail vein at a dose of 1×10¹¹ GC/mouse. Mice were euthanized following a 6 h fast from 9 am to 3 pm. Animals received humane care according to the criteria outlined in the “Guide for the Care and Use of Laboratory Animals.” All animal protocols were approved by the Institutional Animal Care and Use Committee of the University of Oklahoma Health Sciences Center.

Bile Acid Analysis: Total Bile Acid Measurement and LC-MS Method

Bile acids were extracted from liver, whole gallbladder bile, whole small intestine with content, and dried feces in 90% ethanol. To collect fecal sample, an individual mouse was placed in a jar briefly and fresh feces were collected. Total bile acid amount was measured by a bile acid assay kit. Bile acid pool was calculated as the total bile acids in liver, gallbladder and small intestine. Bile acid composition in the gallbladder bile and the pooled fecal extracts was analyzed on a Thermo Scientific UltiMate 3000 UHPLC with a Waters Cortecs C18 column and a TSQ Quantis triple quadrupole mass spectrometer. Standard curves for bile acids and internal standard glycine-CDCA-d4 were generated with purified compounds and relative area under the curve (AUC) was calculated. To measure T-CDCA-d4 metabolism in fecal slurry mixtures, pooled fresh fecal sample from each group was resuspended in a reaction buffer consisting of 10% PBS (pH=7.4) and 90% 3 mM sodium acetate (pH=5.2) to a final suspension of 4 mg fecal sample/ml. T-CDCA-d4 was added to a final concentration of 20 ug/ml and the mixture was incubated at 37° C. for 6 h. An equal amount of methanol was added and the mixture was incubated on ice for 1 h to precipitate protein. After centrifugation, an aliquot of the supernatant was vacuum dried and resuspended in injection buffer (0.1% formic acid, 30% acetonitrile, 70% HPLC water), which was used for LC-MS measurement of bile acids as described above.

Liver and Intestine Histology and Immunohistochemistry

Liver tissues and colon tissues were fixed in 4% paraformaldehyde and paraffin embedded. Hematoxylin and Eosin (H&E) staining was performed with an automated stainer. Sirius Red staining was performed with Direct Red 80 solution (Sigma #365548, St. Louis, MO). For immunohistochemistry, paraffin embedded tissues were deparaffinized and rehydrated, and after antigen retrieval, 5 m sections were incubated first with blocking buffer (5% BSA and 5% goat serum in PBS) for 1 h and then blocking buffer containing primary antibodies overnight at 4° C. After washing with PBS, the sections were incubated with secondary antibodies in SignalStain® Boost IHC Detection Reagent (Cell Signaling, #8114, Danvers, MA) for 1 h. Signal was then visualized using a DAB kit (Cell Signaling, #11724, Danvers, MA). The sections were counterstained with Hematoxylin. Images are acquired by using an EVOS M5000 imaging system (ThermoFisher Scientific, Grand Island, NY).

Real-Time PCR

Total liver RNA was purified with Trizol (Sigma-Aldrich, St. Louis, MO). Reverse transcription was performed by using Oligo dT primer and SuperScript III reverse transcriptase (ThermoFisher Scientific, Grand Island, NY). Real-time PCR was performed on a Bio-Rad CFX384 Real-time PCR system with iQ SYBR Green Supermix (Bio-Rad, Hercules, CA). 18S was measured and used for normalization. The comparative CT (Ct) method was used to determine the relative mRNA expression with the control group arbitrarily set as “1”.

Statistical Analysis

All results were expressed as mean±SEM. One-way ANOVA followed by Tukey post hoc test or Student's t-test was used to calculate the p value. A p<0.05 was considered statistically significant.

Results

Cyp2c70 KO mice show human-like hydrophobic bile acid pool and cholangiopathy and portal fibrosis

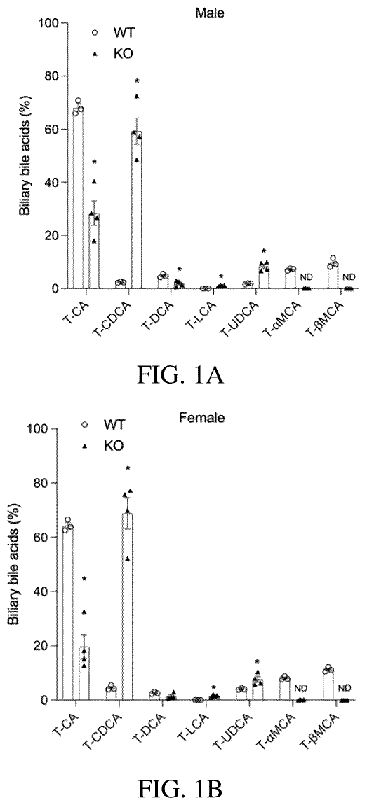

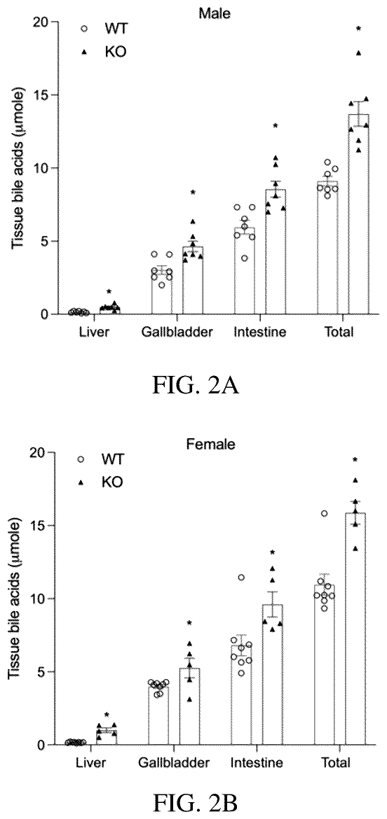

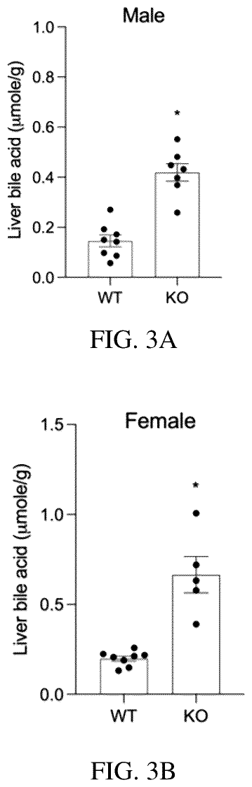

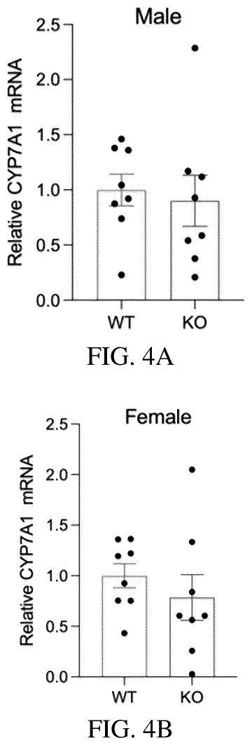

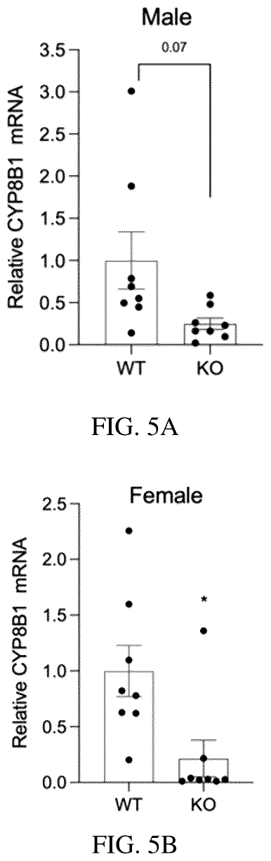

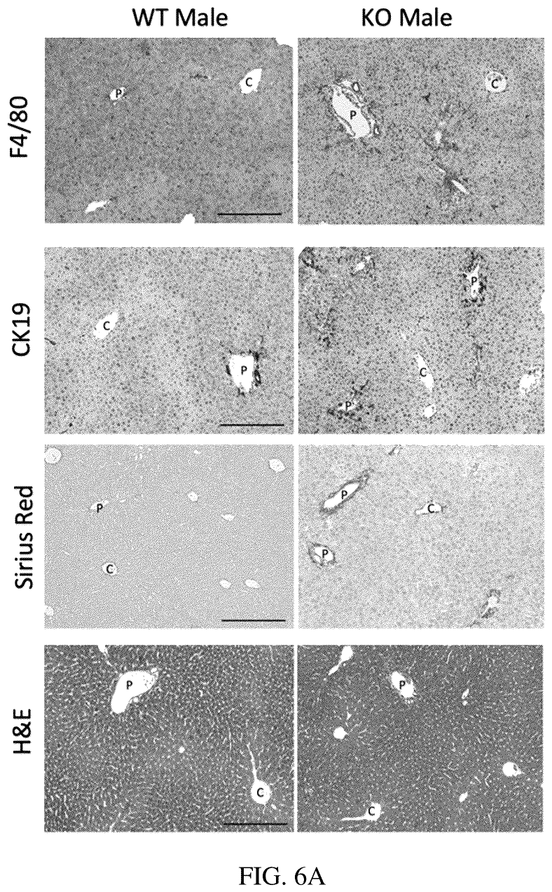

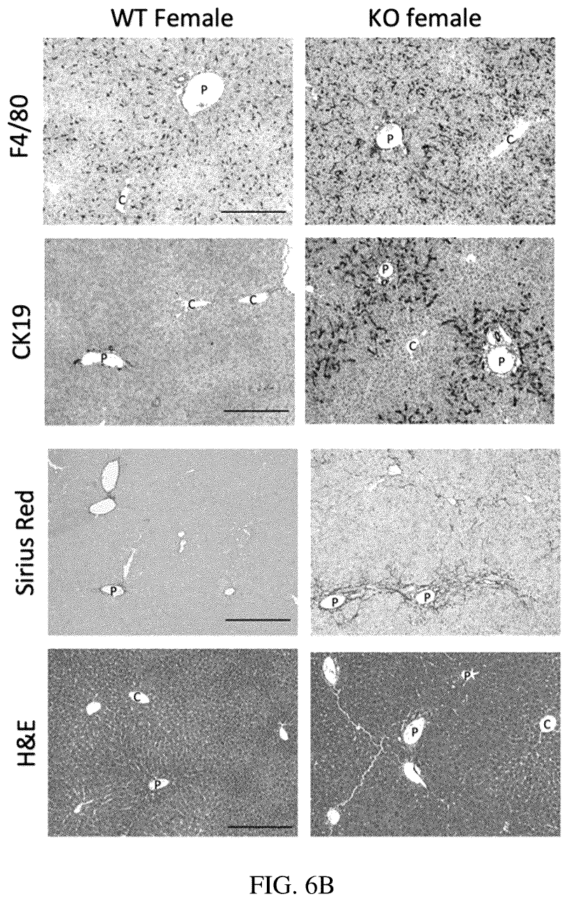

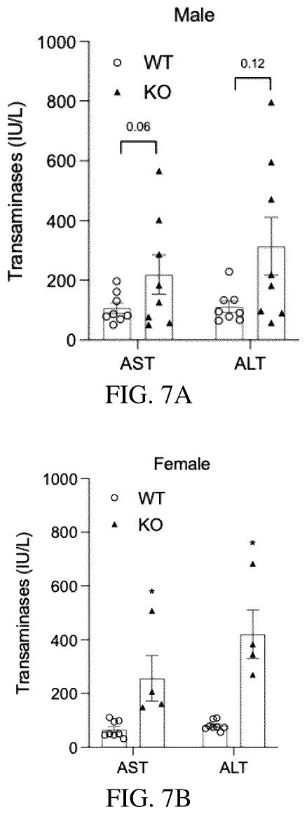

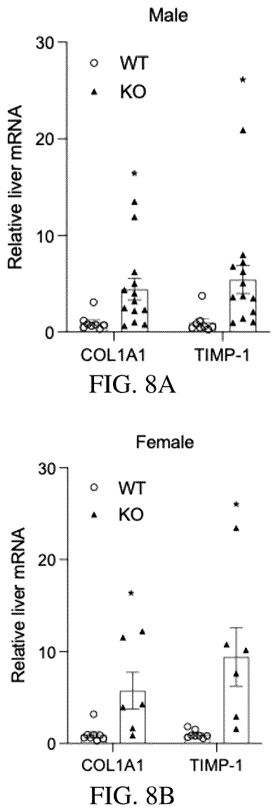

Genetic deletion of the Cyp2c70 gene resulted in complete absence of MCAs in the gallbladder bile of both male and female Cyp2c70 KO mice (FIG. 1 (A-B)). Due to the lack of CYP2C70-mediated conversion of CDCA to MCA, the gallbladder bile acid pool in both 16 weeks old male and female Cyp2c70 KO mice consisted primarily of ˜60-70% tauro-conjugated CDCA (T-CDCA) and ˜20-30% T-CA. Because CDCA can be converted to UDCA via 7-HO epimerization and CYP2C70 oxidizes UDCA to MCA, the lack of CYP2C70 also resulted in ˜8% T-UDCA in the bile of the Cyp2c70 KO mice, which was higher than the ˜2-4% of T-UDCA found in the bile of the WT mice (FIG. 1 (A-B)). Consistent with increased T-CDCA, the bile acid pool of the Cyp2c70 KO mice contained ˜1-1.5% of tauro-lithocholic acid (T-LCA) compared to ˜0.04% of T-LCA found in the gallbladder bile of the WT mice (FIG. 1 (A-B)). Similarly, the bile acid pool of the Cyp2c70 KO mice showed significantly lower T-DCA abundance (˜1-3%) than the WT mice, correlating with reduced T-CA abundance than the WT mice (FIG. 1 (A-B)). The unconjugated bile acids collectively accounted for less than 1% of the overall bile acid pool and were not further analyzed. In addition to altered bile acid composition, the Cyp2c70 KO mice also showed significantly increased bile acid content in the liver, gallbladder and small intestine, resulting in a ˜50% larger bile acid pool in both male and female Cyp2c70 mice than the WT mice (FIG. 2 (A-B)). Consistently, hepatic bile acid concentration was significantly increased by ˜3-fold in the Cyp2c70 KO mice than the WT mice (FIG. 3 (A-B)). Despite a larger and more hydrophobic bile acid pool, the Cyp2c70 KO mice showed similar liver CYP7A1 mRNA compared to the WT mice (FIG. 4 (A-B)). However, the mRNA of sterol 12a-hydroxylase (CYP8B1) was ˜80% lower in the Cyp2c70 KO mice than the WT mice (FIG. 5 (A-B)), which provided an explanation of reduced T-CA abundance in the Cyp2c70 KO mice (FIG. 1 (A-B)). At 16 weeks of age, both male and female Cyp2c70 KO mice showed increased inflammatory infiltration (F4/80 and H&E stain), ductular reaction (CK19 stain) and portal fibrosis (Sirius Red stain) (FIGS. 6A-6B). Liver injury and portal fibrosis were consistent with elevated serum aspartate aminotransferase (AST) and alanine aminotransferase (ALT) (FIG. 7 (A-B)) and hepatic mRNA expression of COL1A1 and TIMP-1 in the Cyp2c70 KO mice (FIG. 8 (A-B)). However, the female Cyp2c70 KO mice showed more severe inflammatory infiltration, ductular reaction and portal fibrosis than the male Cyp2c70 KO mice (FIGS. 6A-6B). In summary, the Cyp2c70 KO mice showed human-like hydrophobic bile acid pool composition resulting in female-predominant cholangiopathy and portal fibrosis.

Combined ASBT Inhibitor and AAV-FGF15 Treatment Effect on Portal Fibrosis

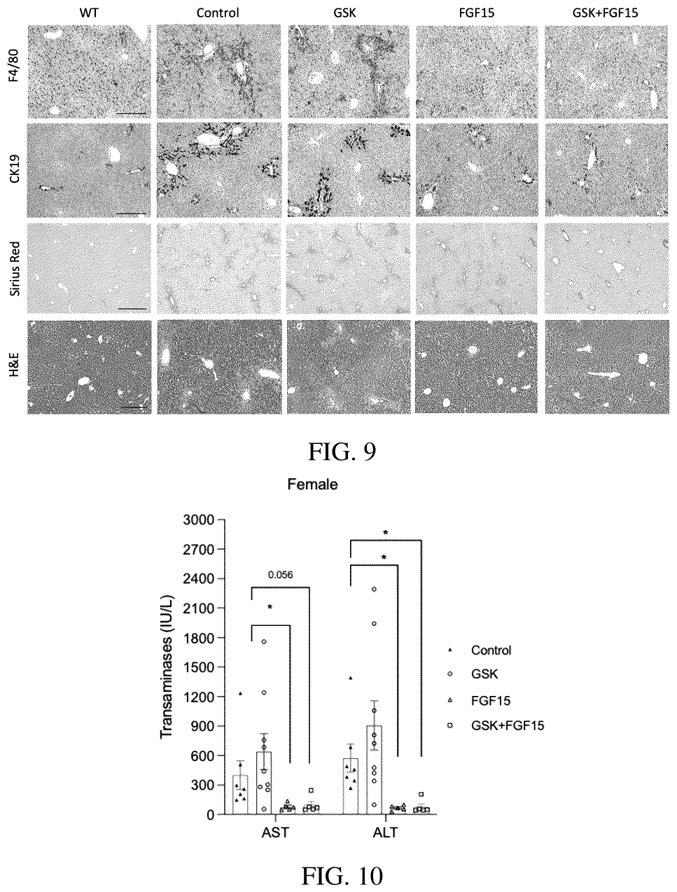

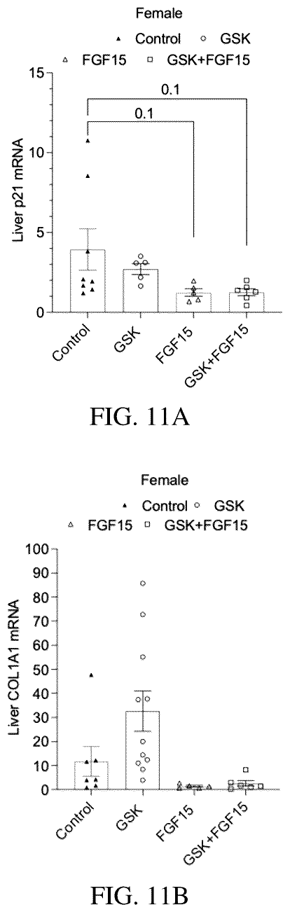

We next compared the therapeutic benefits of a gut-restricted ASBT inhibitor GSK, AAV-FGF15, and the combination of the two treatments in modulating bile acid pool size and composition and liver injury in the Cyp2c70 KO mice. Combined ASBT inhibitor and AAV-FGF15 treatment reversed biliary injury and portal fibrosis in the female Cyp2c70 KO mice. In the female Cyp2c70 KO mice with more severe liver injury, after 4 weeks of treatment initiated when mice were 12 weeks of age, the GSK treatment was largely ineffective in alleviating portal inflammation, ductular reaction or fibrosis (FIG. 9). The AAV-FGF15 treatment significantly decreased portal inflammatory infiltration and ductular reaction but was less effective in reversing portal fibrosis (FIG. 9). In comparison, the combined treatment largely decreased portal inflammation, ductular reaction and portal fibrosis (FIG. 9). Consistent with these liver histopathological improvements, serum transaminases were reduced by AAV-FGF15 and the combined treatment but was not altered by the GSK treatment (FIG. 10). The mRNA expression of liver fibrogenesis marker COL1A1 trended lower in the AAV-FGF15 treatment and the combined treatment groups, but not the GSK treatment group, although these changes did not reach statistical significance due to relatively large variations (FIG. 11(A)). Cellular senescence has recently been identified as a hallmark feature of biliary injury. GSK treatment did not affect liver p21 mRNA expression, while both the AAV-FGF15 treatment and the combined treatment reduced liver p21 mRNA by ˜70% (FIG. 11(B)), which closely correlated with reduced inflammatory infiltration and ductular reaction in these mice. In summary, both the AAV-FGF15 treatment and the combined treatment decreased portal inflammation and ductular reaction, but only the combined treatment achieved reversal of portal fibrosis in the female Cyp2c70 KO mice.

Combined ASBT inhibitor and AAV-FGF15 treatment reduced bile acid pool

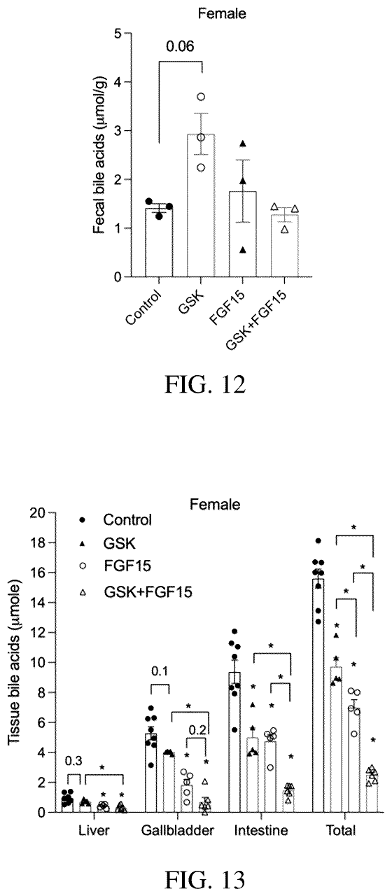

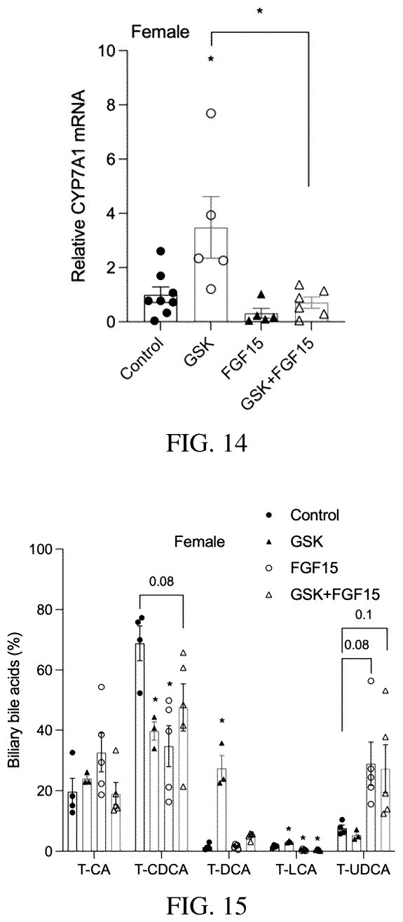

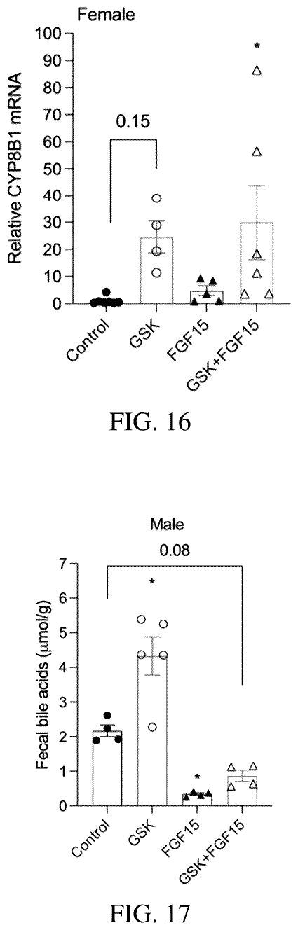

To understand the underlying mechanisms associated with the observed therapeutic effects, we next studied bile acid metabolism in Cyp2c70 KO mice. Combined ASBT inhibitor and AAV-FGF15 treatment reduced bile acid pool reduction and enriched T-UDCA. Fecal bile acid measurements revealed that the GSK treatment increased fecal bile acid loss by ˜2-fold after 2 weeks treatment, the AAV-FGF15 treatment did not alter fecal bile acid excretion after 2 weeks but its presence in the combined treatment prevented induction of fecal bile acid excretion by GSK (FIG. 12). After 4 weeks of treatment, analysis of the bile acid pool revealed that although all three treatments decreased tissue bile acid content and total bile acid pool, the GSK treatment and the AAV-FGF15 treatment reduced the total bile acid pool by ˜40% and ˜50%, respectively (FIG. 13). In comparison, the combined treatment reduced the total bile acid pool by ˜80% (FIG. 13), which may be explained by the lack of hepatic CYP7A1 induction in the presence of GSK and maintained fecal bile acid excretion despite a smaller bile acid pool compared to WT mice (FIGS. 12-14). Analysis of gallbladder bile acid composition further revealed that GSK treatment decreased T-CDCA abundance from −70% to ˜40% and increased T-DCA abundance from ˜1% to ˜30% without affecting T-CA abundance compared to the untreated Cyp2c70 KO mice (FIG. 15). This may be explained by markedly increased hepatic CYP8B1 that shifted de novo bile acid synthesis towards CA production over CDCA production (FIG. 16), while some of the CA was efficiently converted to DCA in the gut. However, given that T-CDCA and T-DCA have very similar hydrophobicity index, these GSK-dependent bile acid composition changes may not markedly alter the overall bile acid pool hydrophobicity.

Interestingly, the AAV-FGF15 treatment did not affect T-CA or T-DCA abundance but decreased T-CDCA abundance from −70% to ˜50% and increased T-UDCA abundance from −8% to ˜30% compared to the untreated Cyp2c70 KO mice (FIG. 15), suggesting that AAV-FGF15 somehow promoted T-CDCA conversion to T-UDCA in the female Cyp2c70 KO mice. This bile acid composition change is expected to result in a less hydrophobic bile acid pool, which may partially explain why only the AAV-FGF15 treatment, but not the GSK treatment, attenuated portal inflammation and ductular reaction although both treatments reduced the total bile acid pool by a similar magnitude. Although the combined treatment also increased hepatic CYP8B1 expression as the GSK treatment did, the bile acid composition changes resembled that of the AAV-FGF15 treatment group with ˜30% T-UDCA but only modestly increased T-DCA abundance (FIGS. 15-16). The ˜80% reduction of total bile acid pool together with increased T-UDCA abundance in the bile may contribute to the reversal of cholangiopathy and portal fibrosis in the combined treatment group.

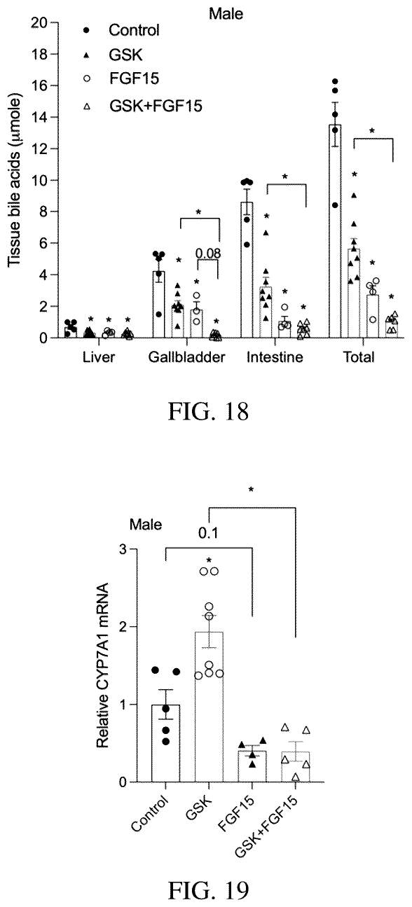

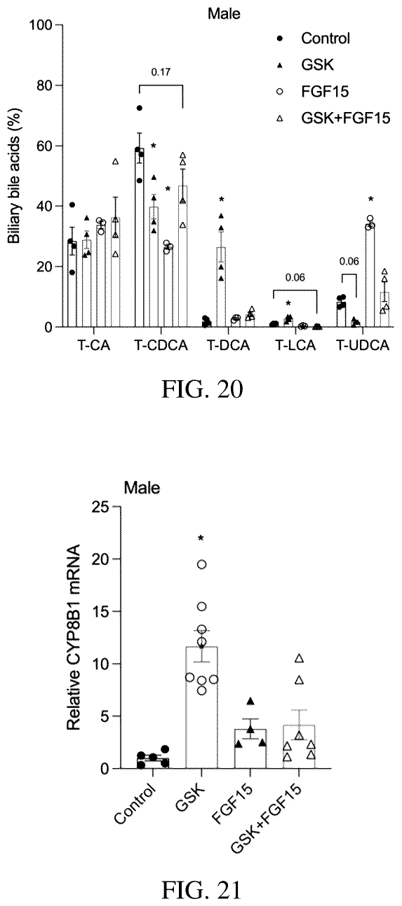

The combined ASBT inhibitor and AAV-FGF15 treatment reduced bile acid pool size in the male Cyp2c70 KO mice. Further analysis revealed that the GSK treatment increased fecal bile acid excretion while the AAV-FGF15 treatment and the combined treatment decreased fecal bile acid excretion after 2 weeks of treatment initiation (FIG. 17). After 4 weeks of treatment, the GSK treatment decreased bile acid pool size by ˜60%, the AAV-FGF15 treatment reduced bile acid pool size by ˜80%, while the combined treatment reduced the bile acid pool size by more than 90% in the male Cyp2c70 KO mice (FIG. 18). The GSK treatment and the AAV-FGF15 treatment also resulted in similar bile acid pool composition changes in the male Cyp2c70 KO mice as they did in the female Cyp2c70 KO mice, with the GSK treatment increasing T-DCA abundance explained by CYP7A1 and CYP8B1 induction (FIGS. 19-21) and the AAV-FGF15 treatment increasing T-UDCA abundance to ˜35% of the total bile acid pool (FIG. 20). In the combined treatment group, there was only a trend towards reduced T-CDCA abundance and increased T-UDCA abundance, but these changes were very modest and statistically insignificant (FIG. 20). Therefore, the combined treatment resulted in more than 90% reduction of the bile acid pool with a composition that was similar to that of the untreated male Cyp2c70 mice but failed to improve cholangiopathy or portal fibrosis.

Combined Treatment Restored Gut Barrier Integrity in the Cyp2c70 KO Mice

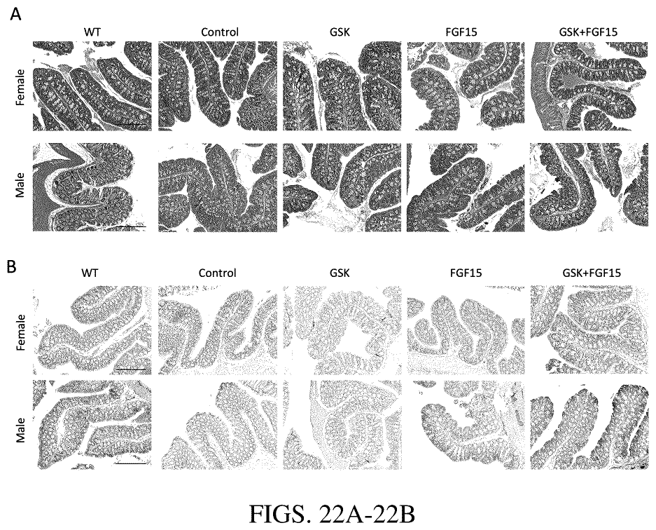

Cyp2c70 KO mice have impaired gut barrier integrity. Bile acids are known to play important roles in maintaining normal gut functions under physiological condition, while exposure to high levels of hydrophobic bile acids under diseased conditions can also impair gut barrier function. Because GSK, AAV-FGF15 and the combined treatment are expected to have differential impact on colonic bile acid flux, we investigated their effect on gut barrier integrity. Initial evaluation of H&E sections by a clinical pathologist did not reveal abnormal histological features of the colon epithelium of both male and female Cyp2c70 KO mice, either untreated or treated with GSK, AAV-FGF15, or the combined treatment (FIG. 22(A)). However, immunohistochemistry staining of the tight junction protein Zonula Occludens-1 (ZO-1) revealed significantly reduced ZO-1 intensity in the colon of both male and female Cyp2c70 KO mice than the WT mice (FIG. 22(B)), suggesting disrupted gut barrier integrity in the Cyp2c70 KO mice. In both male and female Cyp2c70 KO mice, the GSK treatment failed to restore ZO-1 level. The AAV-FGF15 treatment restored ZO-1 level in the male Cyp2c70 KO mice but not the female Cyp2c70 KO mice, while the combined treatment largely restored ZO-1 in both male and female Cyp2c70 KO mice to a level that was comparable to that of the WT mice (FIG. 22(B)).

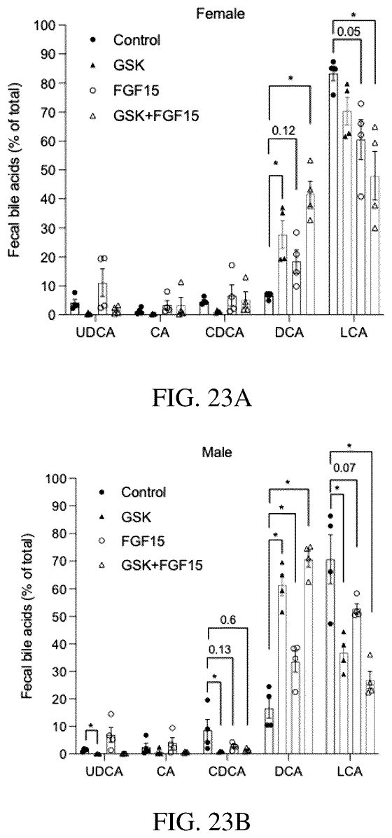

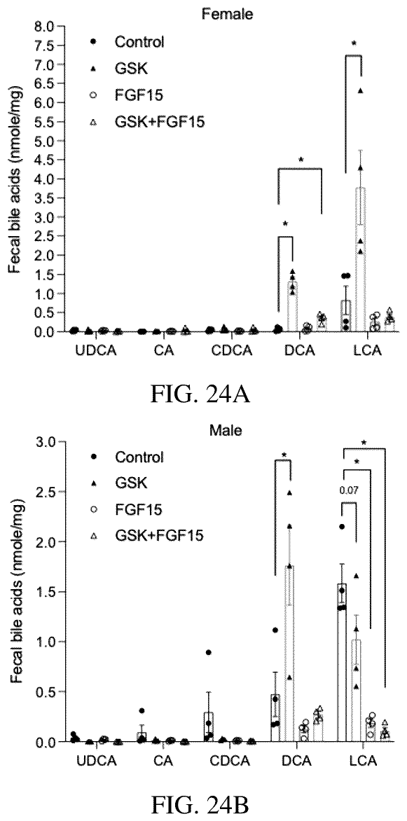

To understand how altered gut bile acid metabolism may contribute to the restored gut barrier integrity in the combined treatment group, we analyzed fecal bile acid composition, which has been shown to closely reflect colonic bile acid composition after microbial transformation of bile acids. We found that LCA accounted for ˜90% of total fecal bile acids with very low abundance of DCA, UDCA, CA and CDCA and largely undetectable conjugated bile acids in the untreated female Cyp2c70 KO mice (FIG. 23(A)). The GSK treatment slightly reduced the LCA abundance and increased DCA abundance (FIG. 23(A)), which may be due to decreased T-CDCA, the precursor of LCA in the gut, and increased abundance of T-DCA in the gallbladder bile of the treated mice. Likely because of reduced biliary T-CDCA, both AAV-FGF15 treatment and the combined treatment reduced fecal LCA abundance, which also resulted in relatively higher abundance of other bile acids in the fecal samples (FIG. 23(A)). In addition, the absolute amount of the fecal LCA and DCA were higher in the GSK-treated mice than the untreated mice due to increased overall fecal loss (FIG. 24(A)). In contrast, the fecal LCA amount was modestly lower in the AAV-FGF15 group and markedly lower in the combined treatment group compared to the untreated mice (FIG. 24(A)), suggesting much lower flux of LCA through the colon of these mice. The male Cyp2c70 KO mice showed similar fecal bile acid composition as the female Cyp2c70 KO mice under untreated condition (FIG. 23(B)). GSK treatment did not increase fecal LCA amount and increased fecal DCA amount (FIG. 24(B)), which correlated with increased biliary T-DCA abundance. The AAV-FGF15 treatment decreased both fecal LCA and DCA amount and thus did not alter the fecal bile acid composition compared to untreated mice (FIG. 23(B)-24(B)). In contrast, the combined treatment reduced fecal LCA but not fecal DCA (FIG. 24(B)), resulting in reduced relative abundance of LCA in the fecal samples (FIG. 23(B)).

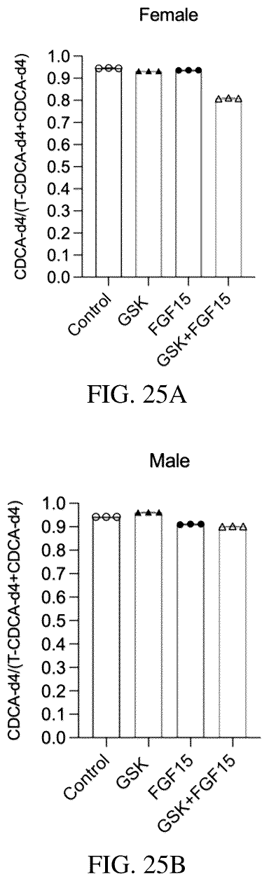

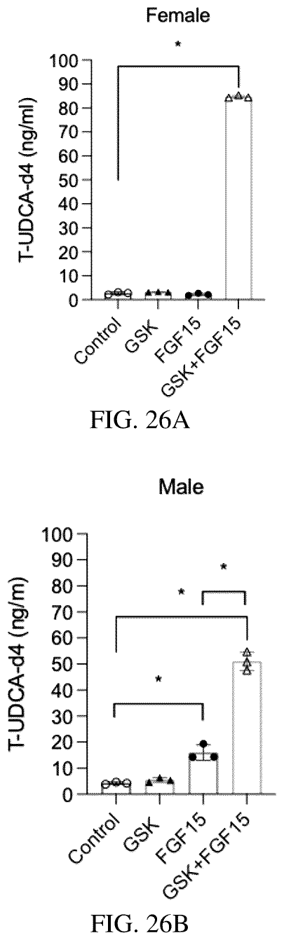

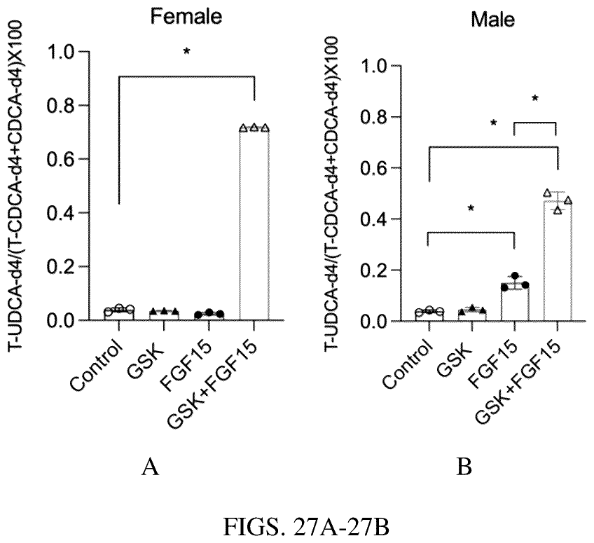

CDCA epimerization sequentially catalyzed by gut microbial 7ca-hydroxysteroid dehydrogenase (7(α-HSDH) and 7 β-hydroxysteroid dehydrogenase (7β-HSDH) produces UDCA in humans and conventionally raised mice, while germ-free mice were also able to produce T-UDCA via mechanisms independent of gut microbiome. However, the fecal bile acid composition is markedly different from the hepatobiliary bile acid composition due to the microbial transformation of bile acids and poorly correlates with biliary bile acid composition (FIGS. 15, 20, 23, 24). We also found that the fecal bile acid composition did not show consistent UDCA enrichment correlating with increased T-UDCA enrichment in the gallbladder bile of the AAV-FGF15 treated male and female mice and the combined treatment group of female mice (FIGS. 15, 20, 23, 24). Therefore, we next incubated T-CDCA-d4 with pooled fecal slurry from each group of mice and measured the production of CDCA-d4, UDCA-d4 and T-UDCA-d4 over a 6-h period that was similar to the colonic transit time in mice. The ratio of CDCA-d4 to the sum of T-CDCA-d4 and CDCA-d4 suggests highly efficient bile acid deconjugation by bacterial bile salt hydrolase activity in all groups of mice (FIG. 25(A-B)). Interestingly, the net production of T-UDCA-d4 was much higher in the fecal slurry of the combined treatment group of the male and female Cyp2c70 mice (FIG. 26(A-B)). Higher T-UDCA-d4 was also found in the AAV-FGF15 treated male but not female Cyp2c70 KO mice (FIG. 25(A-B)). Interestingly, UDCA-d4 was not detected in any of the fecal slurry mixture despite much higher abundance of CDCA-d4 than T-CDCA-d4(FIG. 25(A-B)), suggesting the possibility that T-CDCA-d4 was the preferred substrate for T-UDCA-d4 production under the in vitro assay condition. However, it was estimated based on the T-UDCA-d4 to T-CDCA-d4+CDCA-d4 ratio that only less than 1% of the added T-CDCA-d4 was converted to T-UDCA-d4 (FIG. 27(A-B)). Taken together, unlike the colon of WT mice that was mainly exposed to DCA and the hydrophilic MCA, the colon of the Cyp2c70 KO mice was predominantly exposed to high levels of LCA, and improved gut barrier integrity by the AAV-FGF15 treatment and the combined treatment closely correlated with reduced LCA exposure in the male and female Cyp2c70 KO mice. In addition, the gut microbiome from the combined treatment group may have higher microbial enzyme activity for T-UDCA production from T-CDCA than that of the untreated Cyp2c70 KO mice.

### DISCUSSION

Human patients with cholestasis benefit from reduction of bile acid pool size and hydrophobicity, which serves as a key rationale for developing and testing several bile acid-based therapeutics for cholestasis treatment. Currently, most studies in experimental rodent models and human clinical trials have mainly focused on bile acid-based monotherapies, while whether combining existing therapies with distinct mechanisms of action can potentially achieve improved therapeutic efficacy has not been well investigated. Furthermore, commonly used WT rodent experimental cholestasis models have a different bile acid pool composition than that of humans. The present disclosure is directed to an investigation of the effects of the gut-restricted ASBT inhibitor GSK, the AAV-FGF15 treatment that mimics the effect of an FGF19 analogue, and the combined treatment on bile acid metabolism and cholangiopathy and portal fibrosis induced by a human-like hydrophobic bile acid pool in the Cyp2c70 KO mice.

In both male and female Cyp2c70 KO mice, the combined treatment was able to achieve a significantly higher degree of bile acid pool reduction than either treatment alone. De novo bile acid synthesis and ileal bile acid uptake are two bile acid-sensing mechanisms that help maintain a relatively constant bile acid pool under normal physiology. However, bile acid synthesis has not been reported to be consistently repressed in all cholestatic conditions in humans and experimental models. Reduced bile acid flow into the intestine in cholestasis may diminish both the intestinal FGF15/19-mediated signaling and fecal bile acid excretion, explaining why the adaptive responses are mostly insufficient to significantly limit bile acid toxicity in cholestasis. Because the novel combined treatment simultaneously limited hepatic bile acid synthesis and intestinal bile acid re-uptake, it achieved a much stronger reduction of bile acid pool than either single treatment. In the female Cyp2c70 KO mice exhibiting severe progressive cholangiopathy and portal fibrosis and thus representing a difficult to treat cholestasis condition, additional reduction of the bile acid pool was likely a major mechanism that contributed to the reversal of cholangiopathy and portal fibrosis by the combined treatment. In contrast, the GSK treatment failed to offer therapeutic benefits, while the AAV-FGF15 treatment only alleviated cholangiopathy but not portal fibrosis in the female Cyp2c70 KO mice.

In both male and female Cyp2c70 KO mice, ZO-1 staining revealed significantly impaired gut barrier integrity. Cholestasis has been associated with impaired gut barrier function, which in turn contributes to liver inflammation via the gut-liver axis. Therefore, changes in the gut barrier integrity may also contribute to liver injury in the Cyp2c70 KO mice. Interestingly, in contrast to the hepatobiliary exposure to T-CDCA enriched bile acid pool, we found that the colon of the Cyp2c70 KO mice was predominantly exposed to the toxic LCA and to much less extent DCA, which correlated with higher abundance of T-CDCA in the bile of these mice. This was different from WT mice with DCA and the hydrophilic MCA being the major bile acids in the fecal samples.

In summary, the present results demonstrate that combining an ASBT inhibitor GSK with AAV-FGF15 treatment can achieve a higher magnitude of reduction of bile acid pool accompanied with increased hepatobiliary T-UDCA enrichment that lowered bile acid pool hydrophobicity. This combined treatment was highly effective in alleviating cholangiopathy and portal fibrosis in the female Cyp2c70 KO mice, indicating that combining an ASBT inhibitor and an FGF19 analogue provides improved therapeutic efficacy in forms of cholestasis that can benefit from reduced bile acid pool.

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the formulations of the various compositions described herein, the methods described herein or in the steps or the sequence of steps of the methods described herein without departing from the spirit and scope of the present disclosure. Further, while various embodiments of the present disclosure have been described in exemplary claims herein below, it is not intended that the present disclosure be limited to these particular exemplary claims.

What is claimed is:

1. A method of treating a cholestasis liver condition in a subject in need of such therapy, comprising: administering to the subject a first compound and a second compound, wherein the first compound is selected from the group consisting of a fibroblast growth factor 19 (FGF19) analogue and a bile acid-activated farnesoid x receptor (FXR) agonist, and the second compound is an apical sodium-dependent bile acid transporter (ASBT) inhibitor.

2. The method of claim 1, wherein the FGF19 analogue is Aldafermin (NGM282).

3. The method of claim 1, wherein the FXR agonist is selected from the group consisting of Obeticholic acid (Ocaliva), Tropifexor, Cilofexor, EDP-305, EDP-297, and MET409.

4. The method of claim 1, wherein the ASBT inhibitor is selected from the group consisting of Linerixibat (GSK2330672), Lopixibat, Volixibat (SHP626), Odevixibat (A4250), Barixibat, Elobixibat (A3309), SC-435, S-1647, IMB17-15, 264W94, S-8921, S-8921G, R-146224, BRL-39924A, and S 0960.

5. The method of claim 1, wherein the cholestasis liver condition is at least one of (1) Primary biliary cholangitis, (2) Primary sclerosing cholangitis, (3) Biliary atresia, (4) Progressive familial intrahepatic cholestasis 1 (PFIC 1), (5) Progressive familial intrahepatic cholestasis 2 (PFIC 2), (6) Progressive familial intrahepatic cholestasis 3 (PFIC 3), and (7) Progressive familial intrahepatic cholestasis 4 (PFIC 4).

6. The method of claim 1, wherein the first compound and the second compound interact synergistically.

7. The method of claim 1, wherein the first compound is administered before, concurrently with, or after the second compound is administered.
